(19) United States
(12) Patent Application Publication
(10) Pub. No.: US 2009/0324726 A1
(43) Pub. Date: Dec. 31, 2009

(54) Non-Viral Gene Therapy Using Chitosan-Containing Nanoparticles

(76) Inventors: Julio C. FERNANDES, Brossard (CA); Françoise WINNIK, Westmount (CA)

Correspondence Address: GOWLING, LAFLEUR AND HENDERSON LLP, 1. PLACE VILLE-MARIE, 37TH FLOOR, MONTREAL, QC, CA

(21) Appl. No.: 12/434,161

(22) Filed: May 1, 2009

Related U.S. Application Data
(60) Provisional application No. 61/071,499, filed on May 1, 2008.

(51) Int. Cl. A61K 9/14; C07H 1/00; A61K 31/722

(52) U.S. Cl. 424/489; 536/123.1; 514/55; 977/773

(57) ABSTRACT

The present invention concerns a new drug delivery system and more particularly, a non-viral drug delivery system comprising a polymer according to the following formula and useful for treating a disease characterized by over-expression of folic acid receptors on the cell surface.

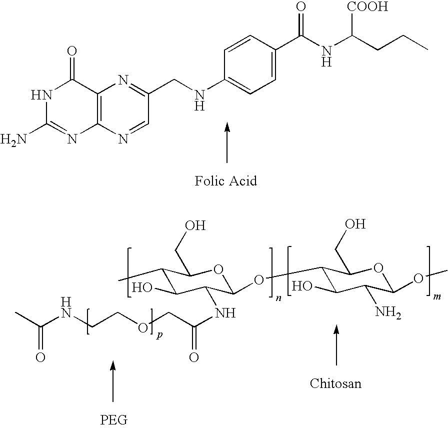

### FIELD OF THE INVENTION

The present invention concerns drug delivery systems and more particularly non-viral drug delivery systems.

### BACKGROUND OF THE INVENTION

Rheumatoid arthritis (RA) is a disabling, chronic inflammatory disease. In this process, interleukin-1 (IL-1) has the role of a crucial mediator of the inflammatory response [1;2]. Its physiological competitive agent, IL-1 receptor antagonist (IL-1Ra), is proven to act as a powerful inhibitor [3]. Several therapeutic studies on the use of recombinant IL-1Ra, as an agent of external source, proved to have a positive effect towards controlling inflammation and symptoms of RA in animal models and in clinical use [4;5].

Rodent models of RA serve as valuable tools to investigate the underlying mechanisms at all stages of RA [6-8]. Rodent models of RA have been developed in both rats and mice [9]. Other species have also been used, however rodent models are most common, due to cost and homogeneity of the genetic background [10]. Most RA in animals is produced with an inducing agent. Adjuvant arthritis has been used in the evaluation of anti-inflammatory drugs (NSAID's, COX-2 inhibitors) [11;12]. AIA in rats shares many features with human RA including genetic linkage, synovial CD4+ cells and T cell dependence [13]. One of the major differences between the AIA model and human is that the inciting agent is known in the model. Other useful rat models of arthritis are streptococcal cell wall-induced arthritis and collagen induced arthritis [12]. There are clear genetic differences among rat and mouse strains in susceptibility to inflammatory arthritis. In some cases, the responses vary depending on the target antigen, induction protocol, and environmental factors [11;14]. These models suggest that there are many pathways to RA in animals, but there is overlap in the inflammatory processes involved. Perhaps the most important lesson is that the same may apply to humans; an array of stimuli provoking a spectrum of pathogenic responses on a range of susceptible backgrounds results in RA.

Viral vectors (e.g. adenovirus) are very effective in term of transfection efficiency, but they have limitations in vivo, particularly by their safety concern and non tissue-specific transfection. On the other hand, non-viral gene transfer systems are limited by their lower gene transfer efficiency, low tissue specificity and transient gene expression. Small size cationic polymers, such as chitosan (Ch), are promising candidates for DNA transport in non-viral delivery systems. The enabling characteristics of Ch-nanoparticles include biocompatibility, multiple ligand affinity, and a capacity of taking up large DNA fragments, while remaining small in size (<100 nm) [15]. We have already shown that FA-chitosan nanoparticles have better characteristics than chitosan-DNA ones, such as lower toxicity and higher transfection efficiency [16-18].

Our study focus on developing a non-viral gene therapy vector based on chitosan nanoparticles and IL-1Ra gene therapy in an Adjuvant-induced Arthritis (AIA) rat model. Specifically, we examined the human IL-1Ra expression level in rat and its macro- and microscopic effects on the inflammation.

It would therefore be highly advantageous to develop a non-viral drug delivery system.

### SUMMARY OF THE INVENTION

We have discovered a novel nanoparticle chitosan-containing polymer. This polymer can be used as a drug delivery system to treat diseases which are characterized by over-expression of folic acid receptors on a cell surface.

Accordingly in one aspect of the present invention, there is provided a polymer of Formula I:

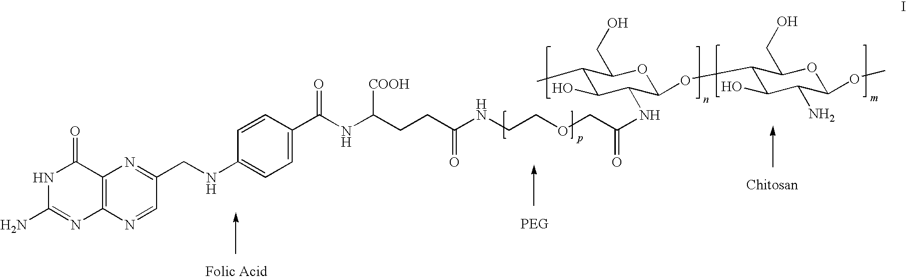

or a salt thereof,
wherein
n is an integer from 1 to 150, preferably 1 to 100, more preferably 1 to 50, more preferably 1 to 20, most preferably about 5 to 6,
m is an integer from 1 to 300, preferably 1 to 250, more preferably 1 to 200, most preferably about 167,
p is an integer from 1 to 200, preferably 1 to 150, more preferably 1 to 100, most preferably about 70; and
wherein the polymer is optionally labeled with a detectable label.

According to another aspect, there is provided a drug delivery system, the system comprising a polymer according to Formula I:

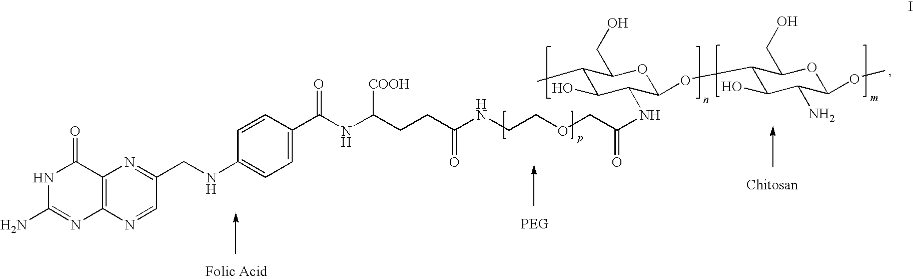

or a salt thereof
wherein
n is an integer from 1 to 150, preferably 1 to 100, more preferably 1 to 50, more preferably 1 to 20, most preferably about 5 to 6,
m is an integer from 1 to 300, preferably 1 to 250, more preferably 1 to 200, most preferably about 167,
p is an integer from 1 to 200, preferably 1 to 150, more preferably 1 to 100, most preferably about 70; and
wherein the polymer is optionally labeled with a detectable label.

According to another aspect of the invention, there is provided a nanoparticle made of a polymer according to Formula I, as described above, the nanoparticle comprising one or more therapeutic agents.

According to another aspect of the invention, there is provided a drug delivery system for administration to a subject, the system comprising a nanoparticle made of a polymer according to Formula I, as described above, the nanoparticle comprising one or more therapeutic agents.

According to another aspect of the invention, there is provided a method of treating a disease-state characterized by over-expression of folic acid receptors on a cell surface, the method comprising: administering to a subject in need thereof, a therapeutically effective amount of a polymer, as described above, so as to treat the disease.

According to another aspect of the invention, there is provided a method of treating a disease-state characterized by over-expression of folic acid receptors on a cell surface, such as an autoimmune disease, the method comprising: administering to a subject in need thereof, a therapeutically effective amount of a polymer, as described above, so as to treat the disease.

According to another aspect of the invention, there is provided a method of treating a disease-state characterized by over-expression of folic acid receptors on a cell surface, such as an autoimmune disease, the method comprising: administering to a subject in need thereof, a therapeutically effective amount of a polymer, as described above, so as to treat the disease and wherein the autoimmune disease is rheumatoid arthritis.

According to another aspect of the invention, there provided a method of treating a disease-state characterized by over-expression of folic acid receptors on a cell surface, the method comprising: administering to a subject in need thereof, a drug delivery system containing a therapeutically effective amount of a polymer, as described above, so as to treat the disease.

According to another aspect of the invention, there provided a method of treating a disease-state characterized by over-expression of folic acid receptors on a cell surface, such as an autoimmune disease, the method comprising: administering to a subject in need thereof, a drug delivery system containing a therapeutically effective amount of a polymer, as described above, so as to treat the disease.

According to another aspect of the invention, there provided a method of treating a disease-state characterized by over-expression of folic acid receptors on a cell surface, the method comprising: administering to a subject in need thereof, a drug delivery system containing a therapeutically effective amount of a polymer, as described above, so as to treat the disease and wherein the autoimmune disease is rheumatoid arthritis.

### BRIEF DESCRIPTION OF THE DRAWINGS

Further aspects and advantages of the present invention will become better understood with reference to the description in association with the following Figures, wherein:

FIG. 1. Coloration of Lewis rats tissue with x-gal. Normal Lewis rats treated with the hydrodynamic intravenous injection wielding 200 μg of β-galactosidase DNA, visualized with X-gal expression 3 days post-injection. (A) Shows the scanned images from the rat soleus muscles. For both groups of tissue samples, we can examine the non-treated controls, the beta-gal (VR1412) nude plasmid injected, the CH-VR1412 nanoparticles injected, and the Ch-FA-VR1412 nanoparticles injected rats. (B) Shows soleus muscle histological cross-sectional patterns of β-galactosidase protein when expression stained with its corresponding antibody after hydrodynamic CH-FA-lacZ injection. Not the mosaic pattern in the position and levels staining within the muscle (Dark brown staining and arrows).

FIG. 2. Decrease in macroscopic inflammation in AIA rat's paws after treatment. Figures represent the average degree of inflammation in rheumatoid arthritis positive control groups with (A) no treatment. Groups treated with Chitosan nanoparticles (B), nude plasmid (C), Chitosan-IL-1Ra (D)

FIG. 3. Rat's ankle size change before-after treatments. Graph shows changes of absolute ankle size (mm) in AIA rats before and after different treatments at day 18. Each point represents the mean±SD. Note a significant decreasing inflammation with Ch-FA-II-1Ra gene therapy (p≦0.05, n=7) comparing to the AIA no-treated control. Differences observed between all treated groups were non-significant (NS).

FIG. 4. Human IL-1Ra ELISA detection. Three rat groups (n=12) were injected with different delivery systems to express human sII-1Ra in sera. Data=means±SD. There is a more sustained expression of IL-1Ra in both chitosan-vectors groups compared to the naked DNA group.

FIG. 5. II-1β concentration in rat sera. All treated groups showed significant decrease IL-1Ra in serum (p≦0.05, n=5) compared to non-treated controls. It can be clearly seen that this effect is observed the day after treatment starts. Black arrow indicates the day of starting IL-1Ra gene therapy.

FIG. 6. PGE₂ level in rat sera after II-1Ra treatment. There is a significant decrease in the levels of PGE₂ in all three treatment groups (*p<0.05, **p<0.01).

FIG. 7. Synthetic procedure of folate-PEG-chitosan.

FIG. 8. ¹H NMR spectrum of folate-PEG-chitosan. Solvent: DCI/D₂O (1:100).

### DETAILED DESCRIPTION OF THE INVENTION

### DEFINITIONS

Unless otherwise stated, the following terms apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “folate over expression” is intended to mean the overexpression of the folic acid cell membrane receptor alpha or beta.

As used herein, the term “a disease-state characterized by over-expression of folic acid receptors on a cell surface” is intended to mean those diseases where this cell membrane receptor is significantly more detectable than in normal states.

As used herein, the term “nanoparticle” is intended to mean a particle that has a diameter or other dimension less than 200 nm, typically less than 100 nm.

As used herein, the term “detectable label” is intended to mean a group that may be linked to the polymer as described herein to produce a probe, such that when the probe is associated with Green Fluorescent Protein or Beta-Galactosidase, the label allows either direct or indirect recognition of the probe so that it may be detected, measured and quantified. Non-limiting examples of a detectable label include radiolabels such as tritium, ¹⁴C, and fluorescent labels.

As used herein, the term “probe” is intended to mean a polymer as described herein which is labeled with either a detectable label or an affinity tag, and which is capable of selectively identifying the cells that were transfected by the nanoparticle system.

As used herein, the term “therapeutic agent” is intended to mean a DNA plasmid, an oligonucleotide, a DNA sequence, a protein, a sequence which induces apoptosis, a drug that induces apoptosis, a biologically active molecule and a drug.

As used herein, the term “affinity tag” is intended to mean a ligand or group, which is linked to a polymer as described herein to allow another compound to be extracted from a solution to which the ligand or group is attached.

As used herein, the term “therapeutically effective amount” is intended to mean an amount of a polymer of Formula I which, when administered to a subject is sufficient to effect treatment for a disease-state characterized by over-expression of folic acid receptors on a cell surface. The amount of the polymer of Formula I will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state characterized by over-expression of folic acid receptors on a cell surface, as disclosed herein, in a subject, and includes: (i) preventing a disease or condition characterized by over-expression of folic acid receptors on a cell surface from occurring in a subject, in particular, when such mammal is predisposed to the disease or condition but has not yet been diagnosed as having it; (ii) inhibiting a disease or condition characterized by over-expression of folic acid receptors on a cell surface, i.e., arresting its development; or (iii) relieving a disease or condition characterized by over-expression of folic acid receptors on a cell surface, i.e., causing regression of the condition.

As used herein, the term “subject” is intended to mean humans and non-human mammals such as primates, cats, dogs, swine, cattle, sheep, goats, horses, rabbits, rats, mice and the like.

We have discovered a novel non-viral gene therapy vector based on chitosan nanoparticles and IL-1Ra gene therapy in an Adjuvant-Induced Arthritis (AIA) rat model. Specifically, we examined the human IL-1Ra expression level in rat and its macro- and microscopic effects on the inflammation. The non-viral gene therapy vector includes chitosan nanoparticles which comprise a polymer of Formula I:

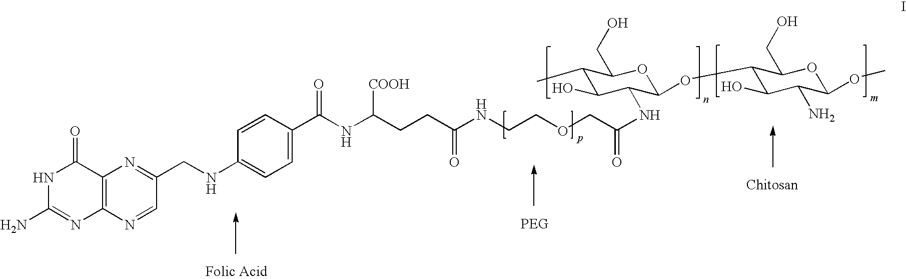

wherein,
n is an integer from 1 to 150, preferably 1 to 100, more preferably 1 to 50, more preferably 1 to 20, most preferably about 5 to 6,
m is an integer from 1 to 300, preferably 1 to 250, more preferably 1 to 200, most preferably about 167,
p is an integer from 1 to 200, preferably 1 to 150, more preferably 1 to 100, most preferably about 70; and
wherein the polymer is optionally labeled with a detectable label.

Alternatively, the chitosan core can be substituted by Poly-ethylene-imine (PEI), or poly-l-lysine (PLL). Diblock co-polymers such as chitosan-HA can also be used.

PEGs of lower molecular weight can also be used. Sialoproteins are also another option for PEG.

The polymer may also be labelled with a detectable label to allow the polymer to be used as a probe or as a research tool. Examples of such applications include, but are not limited to, the delivery of DNA, RNA, siRNA, miRNA, plasmid inside at least one cell expressing folic acid receptor.

This non-viral system could replace virus-based cell transfection systems, with a lower cost and much better safety and user-friendly profile. It would be cell-specific (FA overexpression) or manose-receptor specific (cross-reaction with chitosan).

It could also be used as a labelling tool for FA-overexpressing cells, due to its lower toxicity and higher specificity than liposomes or PEI-based systems.

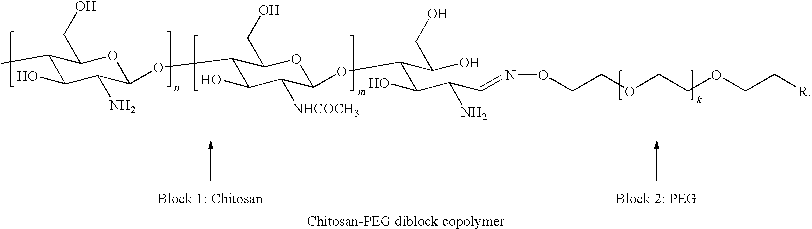

This chitosan-diblock is a new entity, where the whole nanoparticle is assembled in line allowing for better linkage and physical properties.

One particularly useful aspect of our discovery is the use of the aforesaid polymer as a drug delivery vehicle or system to treat disease states, which are characterized by over-expression of folic acid receptors on a cell surface. Examples of such disease states include, but are not limited to, autoimmune diseases such as rheumatoid arthritis and Crohn's disease, Psoriasis, and cancers such as ovarian cancer, breast cancer and lung cancer.

One such disease state is rheumatoid arthritis, which is a chronic inflammatory disease. IL-1 receptor antagonist (IL-1Ra) is a powerful inhibitor of the inflammatory response in animals and humans. The development of effective non-viral vectors is still underway. Using chitosan as a vector, we have previously shown that folic acid (FA)-(polyethylene glycol (PEG)-chitosan nanoparticles are better carriers than chitosan-DNA alone.

Our objective was to develop a non-viral vector based on chitosan nanoparticles and IL-1Ra gene in an Adjuvant-induced Arthritis (AIA) rat model. Arthritis was induced with heat-killed M butyrium suspended in mineral oil injected into the rat's right hind foot. A positive control group (naked DNA), a chitosan-DNA and an FA-PEG-chitosan-DNA nanoparticles groups (N=12) were injected hydrodynamically in the left posterior paw 18 days post-AIA induction (for maximum disease progression). A non-treated group (n=5) was used as a negative control. Groups were compared using t test and one-way ANOVA. Human IL-1Ra was present in rat's blood. Treated animals also showed a significant decrease in paw inflammation, II-1β and PGE₂ compared to untreated rats. Even without significant differences between nanoparticles efficacy, this study demonstrated the usefulness of a non-viral therapeutic approach using hydrodynamic delivery of IL-1Ra and chitosan-DNA based nanocarriers to decrease inflammation in an AIA rat model.

Presence of Nanoparticles in the Soleus Muscle

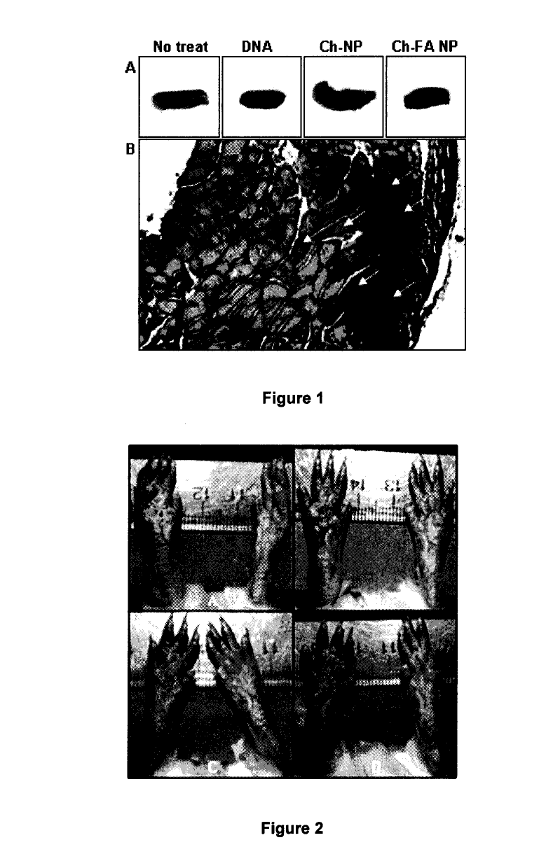

According to FIG. 1, the staining shows the presence of β-gal in the muscle previously injected with a nanoparticle but the protein was absent in the untreated RA group. Therefore, considering that IL-1Ra was delivered using the same nanoparticle and hydrodynamic delivery systems, it indirectly suggests that IL-1Ra is delivered to the muscle.

Macroscopic Inflammation Measurements

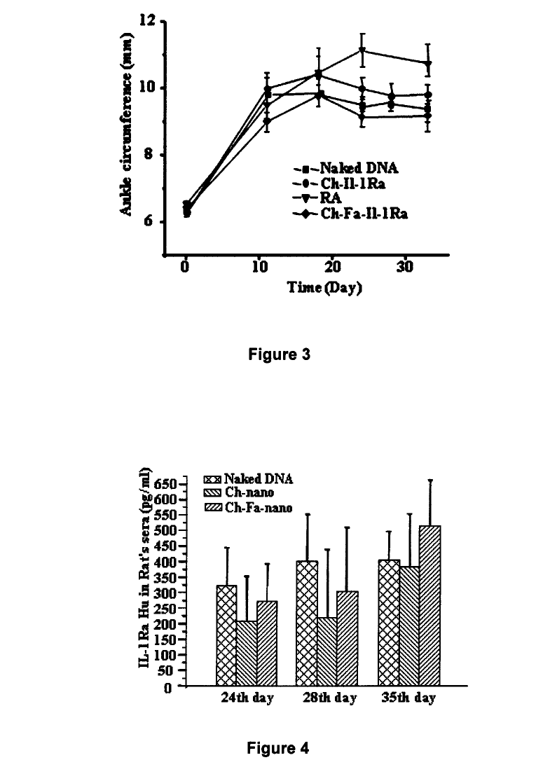

Decrease in macroscopic inflammation in AIA rat's paws after treatment with Chitosan-II-1Ra (Ch-II-1Ra), or Chitosan-PEG-FA-II-1Ra (Ch-FA-II-1Ra) present in various degrees as shown in FIG. 2. According to FIG. 3, there is a significant decrease in the percentage of ankle size in all treated groups compared to the untreated RA positive control group (p<0.05, n=5). Only the Ch-FA-II-1Ra group showed significant decrease in ankle size. However, there was no significant difference between all three treatment groups (naked DNA, Ch alone and Ch-FA).

Human IL-1Ra ELISA Detection

Concentration of human II-1Ra in sera of rats treated with nanoparticles of Ch-IL-1Ra, and Ch-FA-IL-1Ra presented significant differences compared to control group. These results show absolute sIL-1Ra normalized to the measurement of RA group's sera (FIG. 4). As shown in FIG. 4, the naked plasmid is more rapidly expressed in greater quantity in the serum than the Ch-nano and Ch-FA-nano particles. However, despite the longer time taken by Ch nanoparticles to express themeselves in the serum, there were no significant differences in the IL-1Ra Hu concentration across the three treatment groups (naked DNA, Ch alone and Ch-FA).

II-1β Concentration in Serum

To measure the inflammation changes after treatment, cytokine IL-1β, a potent immuno-modulator which mediates a wide range of immune and inflammatory responses, was detected in the serum of the experimental animal rat model. A gradual increase of cytokine level was seen in the positive control group (RA), while there was an abrupt decrease of IL-1β levels following treatment with Ch-IL-1Ra and Ch-FA-IL-1Ra nanoparticles. This brings to the fore the inflammation reduction, since IL-1Ra competes for the IL-1 receptor and indirectly decreases IL-1β production therein (FIG. 5).

PGE₂ Synthesis after IL-1Ra Treatment

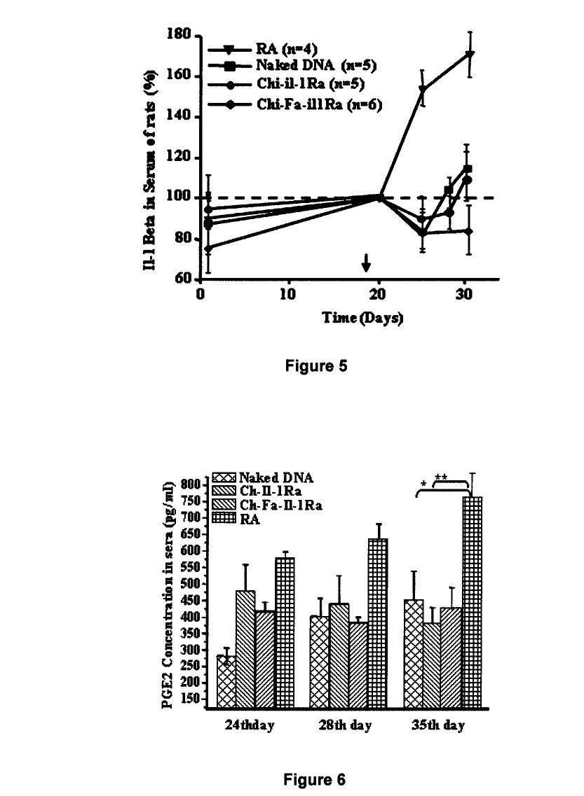

As shown in FIG. 6, a gradual increase of PGE₂ levels is seen in the positive control group (RA), whereas there is a decrease of PGE₂ levels following treatment with Ch-IL-1Ra. Lower levels of PGE₂ synthesis were observed in all treated groups compared to positive control.

Discussion

Our results showed that it is possible to express the IL-1Ra protein with a variety of nanoparticles. Macroscopically, hydrodynamic injection of these nanoparticles in an AIA rat model allow a significant decrease of the inflammation in the rats' ankle compared to untreated rats, proving indirectly the efficacy of the IL-1Ra protein treatment. Microscopically, various inflammation markers (IL-1s and PGE₂) showed a significant decrease in the muscle and serum after the injection of the IL-1Ra protein demonstrating by direct evidence the efficacy of the administration technique to deliver efficient nanoparticles. Therefore, it is possible to do gene therapy with IL-1Ra to decrease arthritis and have a positive effect on inflammation. However, our results have not allowed the demonstration that a nanoparticle had a superior effect to others because they all showed similar patterns of results.

Efficacy of the Hydrodynamic Delivery Technique

The high effective delivery of naked plasmid into body or tissue with hydrodynamic injection technique have already been demonstrated [19;20]. However the mechanism of hydrodynamic injection aiding the delivery of nanoparticles into tissue or reaching its aim in tissue is unclear. In this study, we found that the expression of the targeted protein by a Ch-nanoparticle is possible (as reflected by the staining by β-gal in the soleus muscle) but it takes a longer time before reaching a high level than with naked DNA. Although hydrodynamic injection causes edema at the site of puncture (data not show), injection skills may affect the effect of genetic material delivered [21]. However, in our study, this manipulation was performed by the same person, used to the technique, thereby reducing the effect of the learning curve on the quality of delivery.

In addition, the hydrodynamic injection allows the introduction of high volume solution into the muscle tissue. This situation allows for a greater concentration of nanoparticles around cells suggesting a greater potential of capture through endocytosis. However, our results demonstrated that the increased concentration of Ch-nanoparticles in the cells is slower than with naked DNA. It may be because nanoparticles used in this study are bigger in size (108-180 nm) than naked plasmids. Many researchers [22-24], including us [16], are actually working on the synthesis of smaller size nanoparticles. It may also be because the pressure of the injection provokes leakage of nanoparticles into surrounding tissues or in the blood stream due to extravasation or rupture of the access vein.

Macroscopic Results

Although our results showed that gene therapy with IL-1Ra seems to give significant and measurable results in terms of inflammation decrease in an AIA rat model, these effects are discreet. As a matter of fact, results between treated groups are very similar and when one nanoparticle seems to detach itself from another, it is only toward a significant trend. Two reasons may explain this situation. First, the number of study groups and the low number of animals per groups may have decreased the statistical power of the study to detect highly significant differences between groups. As in the clinic treatment of rheumatoid arthritis by allowing maximum inflammation to occur at day 17 before treatment, lesions including articular fibrosis and chronic edema and swelling might not respond significantly to treatment. We are presently performing a similar study using a prophylactic approach, e.g. starting IL-1Ra gene therapy at day 1 of inducing AIA in the model. Finally, although Ch and Ch-FA are regularly used to form nanoparticles[25], it may not be the most efficient nanoparticle to deliver IL-1Ra in an AIA rat model. This could be due to a possible low disassembling rate between the vector and the plasmid, or a low expression of folic acid receptors by macrophage.

Conclusion.

This study demonstrated the efficacy of a therapeutic approach using hydrodynamic delivery of plasmid IL-1Ra DNA and chitosan-DNA based nano-carriers in controlling the progression of inflammation in an AIA rat model. However, without significant differences between nanoparticles efficacy, more research is needed toward the search for smaller and more performing nanoparticles in the treatment of arthritis using gene therapy.

### Materials and Methods

1. Synthesis of Folate-PEG-Chitosan Conjugate

Chitosan (Ch) (Wako-10, degree of deacetylation (DD) 85%, M_{w}=57 kDa) was purchased from Wako Chemicals USA (Richmond, Va.). Folic acid (FA) was purchased from Sigma-Aldrich Chemical (St-Louis, Mo., USA). α-amino, ω-carboxyl poly(ethylene glycol) (NH₂-PEG-COOH, M_{w}=3,400 Dalton) was purchased from Shearwater Inc (USA). Chitosan was further deacetylated by treating with concentrated NaOH solution (50%) to obtain ˜96% deacetylation degree according to the reported procedure [26]. To prepare the folate-PEG-chitosan conjugate, folic acid was first attached to NH₂-PEG-COOH via the well known carbodiimide chemistry to obtain folate-PEG-COOH. Then the folate-PEG-COOH was again activated by N,N′-Dicyclohexylcarbodiimide (DCC) and N-hydroxysuccinimide (NHS) to convert to the reactive intermediate folate-PEG-CO-NHS and subsequently grafted onto chitosan to achieve the folate-PEG-chitosan conjugate [27]. The level of folate-PEG incorporation was determined to be 1.1 mol % with respect to the glucosamine unit of chitosan by UV-Vis spectroscopy using folic acid as standard (the extinction coefficient (λ_{363 nm}) of folic acid is determined to be 6165 M⁻¹cm⁻¹ in pH 7.4 phosphate buffer (0.1 M)).

2. Plasmid Construction.

We used human interleukin 1 receptor antagonist as a curing agent, with its secretive variant (sIL-1Ra, 177aa, NM_{—}173842 X52015, GenBank). We sub-cloned this gene by PCR with oligos carrying HindIII and XbaI sites (IL-1RaH3CCAAGCTTGAATGGAAATCTGCAGAGGCC (SEQ ID NO: 1), IL-1RaH4 GCTCTAGACTGGGCA GTACTACTCGTCCTCC (SEQ ID NO: 2)), using genomic DNA of macrophage differentiated from monocyte cell line THP1 stimulated with PMA as PCR DNA template. Then PCR product was inserted in the mammalian expression vector pCDNA3 (Invitrogen, Burlington, ON, Canada), that drives over-expression of gene with CMV promoter. To reduce the size of pDNA, the region of PvuII-PvuII was deleted. Final plasmid (3.8 kb) including targeted gene was verified by DNA sequencing. Mass quantity plasmid was prepared with commercial kit (Qiagen, Mississauga, ON, Canada), and finally suspended in sterile water and quantified by UV spectrometer at 260/280 nm.

3. Nanoparticle Synthesis.

0.1% chitosan (Ch) or chitosan-PEG-FA (Ch-Fa) stock solution was prepared in 25 mM acetic acid, stirring continually at 37° C., and then adjusted to pH 5.0 with 1M NaOH. 100 ug/ml DNA (pIL-1Ra) solution was buffered in 43 mM Na₂SO₄[28]. Nanoparticle synthesis was carried out by mixing equal volume of these two stock solutions at room temperature, stirring 30 minutes, and allowing standing one hour before transfection. Two kinds of nanoparticles were synthesised: chitosan-IL-1Ra (Ch-pIL-1Ra) and chitosan-PEG-FA-pIL-1Ra (Ch-Fa-pIL-1RA).

4. Adjuvant-Induced Arthritis (AIA) Rat Model

Arthritis was induced in 150 to 200 g female Lewis rats (Charles Rivers, Montreal, QC, Canada). Briefly, on day 1, rats were anaesthetized with 0.2 ml 0.5% ketamine and 0.5% xylazine hydrochloride, and 0.5 mg of heat-killed M butyrium suspended in mineral oil (5 mg/ml, Difco, Mich., USA) was injected intradermically into the right hind foot. Disease develops around 10-45 days after injection and generally subsides after a month. Arthritis usually develops in all treated animals, as evidenced by dramatic swelling in the injected paws and progressive swelling in all uninjected paws. All animal experiments were approved by the Animal Ethics Committee of the Hôpital du Sacré-Coeur de Montréal.

5. Hydrodynamic Injection of Nanoparticles

The transfection was performed using the hydrodynamic intravenous injection technique in the left posterior paw, as previously published [21;29;30]. Nanoparticle solutions were adjusted to physical osmolarity with 18% sterile NaCl just before injection. The injection point was located in the great saphenous vein inside the leg, in direction of the knee. A tourniquet was located above the knee forcing the solution back into the smaller leg veins and ending up in muscle tissue cells. The 4 ml transfection solution (200 ug DNA) was injected into about 200 g rat, at a speed of 10 ml/minute, with infusion/withdrawal pump Model 940 (Harvard Apparatus, Millis, Mass., USA). The cuff was kept in place for one minute after injection. The eyes were humidified with Liquifilm Tears eyes drops from Allergan (Markham, ON, Canada).

6. Experimental and Control Groups Under Study

A positive control group (naked DNA, n=5), a natural chitosan-DNA nanoparticles group (n=5) and a chitosan FA-PEG -DNA nanoparticles group (n=7) were injected on day 18 post AIA induction. A non-treated group (Rheumatoid arthritis, RA) (n=5) was used as a negative control. All animals were sacrificed on day 35 and clinical results were compared among the groups. The evaluation of reduced adjuvant-induced arthritis was done by evaluating ankle inflammation, decreased articular index scores, ankle circumferences, and sequential calliper measurements of the ankle joints.

7. Histological presence of nanoparticles in the soleus muscle. The verification of the injection solution's pathway was accomplished by replacing the expression cassette of IL-1Ra with the transfected β-galactosidase (β-gal) gene carried by the plasmid VR1412 (VICAL Inc. San Diego, Calif., USA). Afterwards, the results were obtained through X-gal staining (blue coloration) and scanning for the β-galactosidase expression following standard techniques as previously described [31].

8. Blood Samples

While under anesthesia, 7-9 ml of whole rat blood was obtained by heart puncture. Blood was gelled at room temperature then sera were separated by centrifugation at 600 rpm for 10 minutes. Sera were kept separately at −80° C. and then used for the later assays.

9. Immunological Analysis

Human sIL-1Ra detection in rat was carried out with ELISA DuoSet kit (R&D Systems, Minneapolis, Minn., USA). Blood serum samples were diluted 5-10 times in an assay diluent (BioFX, Owings Mills, Md., USA). 100 ul of dilution were added in 96 wells plates. No special treatment was necessary for standard series. The rest of the process followed the kit provider's protocol. Rat PGE₂ and II-1β were detected with ELISA kit (R&D Systems, Minneapolis, Minn., USA), by diluting blood serum samples 100 times with PBS buffer containing 1% BSA.

10. Statistical Analyses

All values are expressed as means±SD and were subjected to t test and one-way ANOVA analysis. A value of 0.05 was considered significant.

11. Synthesis of Folate-PEG-Chitosan Conjugate

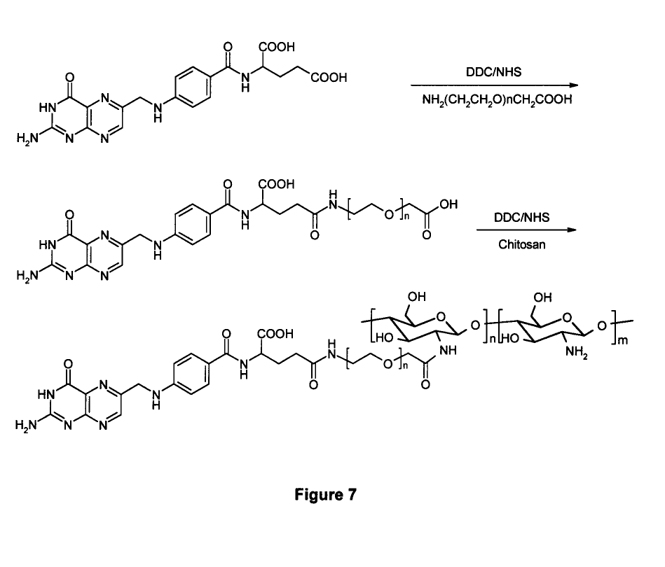

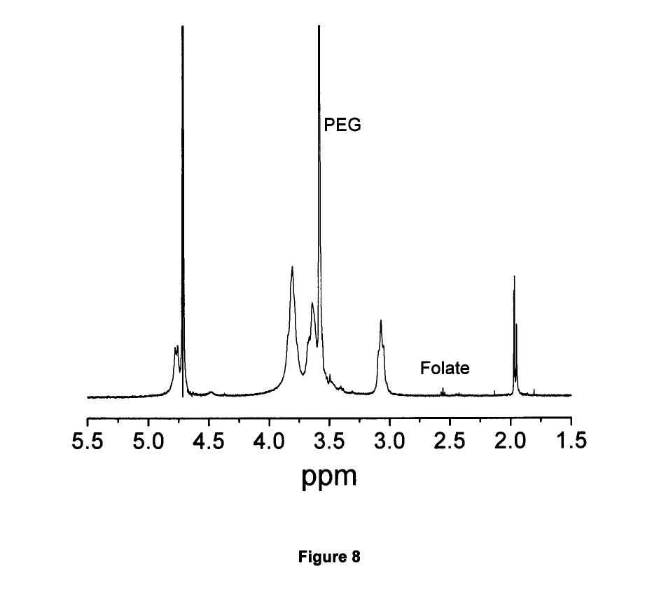

The synthetic scheme of folate-PEG-chitosan conjugate is represented in FIG. 7 (Details on the material, instrumentation and procedure for deacetylation of chitosan as well as ¹H NMR spectrum of folate-PEG-chitosan are provided in FIG. 8). The conjugate was prepared according to the synthetic procedure reported by Cho et al.⁵⁶ with modification. The synthesis, performed by Dr Winnik's team, was accomplished in three steps. 1. folate-NHS Ester: Folic acid (300 mg, 0.67 mmol) was dissolved in 12 mL of dry DMSO to which 93 mg (0.45 mmol) of DCC and 77 mg (0.67 mmol) of NHS were added. The mixture was stirred at room temperature for 6 hours. The white precipitate (a side product of the reaction) was removed by filtration. The filtrate was added to 100 mL of 30% acetone in Et₂O. The resulting yellow precipitate (Folate-NHS ester) was collected by filtration and washed with acetone and ether. It was used immediately in the next step of synthesis. Yield: 250 mg, 83%. 2. folate-PEG-COOH, NH₂-PEG-COOH (450 mg, 0.13 mmol, M_{w}=3,400 Da, Shearwater) was dissolved in 100 mL of dry pyridine. The folate-NHS ester (˜250 mg, 0.55 mmol) was added to the solution and the reaction mixture was stirred overnight in the dark. Upon completion of the reaction, the pyridine was evaporated in vacuo and the remaining solid was dissolved in 20 mL DI water. The product was purified by dialysis against pH 7.4 phosphate buffer (10 mM) for one day and against DI water for two days. Yield 450 mg, 76%. 3. folate-PEG-chitosan. Chitosan (200 mg, 1.25 mmol amino groups) was dissolved in 20 mL of 2% acetic acid aqueous solution. To this solution, folate-PEG-COOH (40 mg, 10 μmol in 5 mL of DMSO and pre-activated by reacting with EDC (2.5 mg, 15 μmol) and NHS (5.8 mg, 25 μmol) for 4 hrs) was added dropwise. The reaction was continued in dark for 20 hrs with constant stirring. Then the reaction mixture was dialyzed using a membrane (MWCO, 12-14 kDa) against deionized water for 3 days. The folate-PEG-chitosan conjugate was recovered by freeze-drying. Yield 210 mg. The level of folate-PEG incorporation was 1.1% with respect to chitosan amino groups, as determined by UV spectroscopy using folic acid as standard (the extinction coefficient (λ_{363 nm}) of folic acid is determined to be 6165 M⁻¹cm⁻¹ in pH 7.4 phosphate buffer (0.1 M)).

Procedure for Deacetylating Chitosan to Synthesise Folate-PEG-Chitosan. Solvent: DCI/D₂O (1:100)

Materials: Chitosan (Wako-10, degree of deacetylation (DD) 85%, M_{w}=57 kDa) was purchased from Wako Chemical Co. N-(3-Dimethylaminopropyl)-N′-ethylcarbodiimide hydrochloride (EDC), N,N′-Dicyclohexylcarbodiimide (DCC), sodium hydroxide (NaOH, 97%), sodium acetate (NaAc), and glacial acetic acid (HAc) were purchased from Sigma-Aldrich Chemical Co. Regenerated cellulose membranes from Spectrum were employed for dialysis. All solvents were of reagent grade and used as received. Water was deionized using a Milli-Q water purification system (Millipore).

Instrumentation: ¹H NMR spectra were recorded on a Bruker ARX-400 400 MHz spectrometer. A DCI/D₂O (1:100, v/v) mixture was used as the solvent for spectra of chitosan. UV/Vis spectra were measured with a Hewlett Packard 8452A photodiode array spectrometer. Gel permeation chromatography (GPC) analysis was carried out on a GPC system consisting of an Agilent 1100 isocratic pump, a Dawn EOS multi-angle laser light scattering detector (MALLS, Wyatt Technology Co.), an Optilab DSP interferometric refractometer (Wyatt Technology Co.), and a TSK-GELPW (Tosoh Biosep, serial number G0014) column. The acetate buffer (acetic acid (0.3M)/sodium acetate(0.2M), pH 4.5) was used as eluent under the condition that injection volume: 100 μL; flow rate: 0.5 mL min.⁻¹; temperature: 25.0° C. The dn/dc values of the polymers were measured at 690 nm with the same refractometer used in the off-line mode. The pH value of sample solutions was determined using an Orion pH-meter.

Deacetylation of Chitosan

The deacetylation of chitosan was carried out according to the reported procedure by Mima, S.; Mararu, M.; Iwamoto, R.; Yoshikawa, S. J. Appl. Polym. Sci. 1983, 28:1909-1917. Briefly, chitosan (4.0 g) was dissolved in 200 mL of aqueous solution of 2% (v/v) acetic acid. The solution was then added dropwise to an aqueous NaOH solution (100 mL, 47 wt %) at room temperature under magnetic stirring. The formed suspension solution was refluxed for 1 hour at 100° C. under N₂ atmosphere. After that, the reaction solution was poured into stirred water (4 L) preheated to 80° C. The precipitate was decanted, filtered, and washed with water for neutralization. The same procedure was repeated once to achieve higher deacetylation degree. The resulting polymer was further purified by dialysis (membrane MWCO of 6-8 kDa) against water for 3 days and isolated by freeze drying (yield 3.2 g, 80%).

### REFERENCE LIST

(1) Fontana A, Hengartner H, Weber E, Fehr K, Grob P J, Cohen G (1982). Interleukin 1 activity in the synovial fluid of patients with rheumatoid arthritis. Rheumatol Int; 2:49-53.

(2) Mizel S B, Dayer J M, Krane S M, Mergenhagen S E (1981). Stimulation of rheumatoid synovial cell collagenase and prostagiandin production by partially purified lymphocyte-activating factor (interleukin 1). Proc Natl Acad Sci USA; 78:2474-2477.

(3) Liao Z, Grimshaw R S, Rosenstreich D L (1984). Identification of a specific interleukin 1 inhibitor in the urine of febrile patients. J Exp Med; 159:126-136.

(4) Schwab J H, Anderle S K, Brown R R, Dalldorf F G, Thompson R C (1991). Pro- and anti-inflammatory roles of interleukin-1 in recurrence of bacterial cell wall-induced arthritis in rats. Infect Immun; 59:4436-4442.

(5) Chang B S, Reeder G, Carpenter J F (1996). Development of a stable freeze-dried formulation of recombinant human interleukin-1 receptor antagonist. Pharm Res; 13:243-249.

(6) Williams R O (1998). Rodent models of arthritis: relevance for human disease. Clin Exp Immunol; 114:330-332.

(7) Williams R O (2004). Collagen-induced arthritis as a model for rheumatoid arthritis. Methods Mol Med; 98:207-216.

(8) Williams R O (2005). Models of rheumatoid arthritis. Ernst Schering Res Found Workshop; 89-117.

(9) Muller-Ladner U, Gay S (1999). The SCID mouse—a novel experimental model for gene therapy in human rheumatoid arthritis. Drugs Today (Barc); 35:379-388.

(10) Robbins P D, Evans C H, Chernajovsky Y (2003). Gene therapy for arthritis. Gene Ther, 10:902-911.

(11) Wooley P H (2004). The usefulness and the limitations of animal models in identifying targets for therapy in arthritis. Best Pract Res Clin Rheumatol; 18:47-58.

(12) Wooley P H (1991). Animal models of rheumatoid arthritis. Curr Opin Rheumatol; 3:407-420.

(13) Goodson T, Morgan S L, Carlee J R, Baggott J E (2003). The energy cost of adjuvant-induced arthritis in rats. Arthritis Rheum; 48:2979-2982.

(14) Griffiths M M (1988). Immunogenetics of collagen-induced arthritis in rats. Int Rev Immunol; 4:1-15.

(15) Sato T, Ishii T, Okahata Y (2001). In vitro gene delivery mediated by chitosan. effect of pH, serum, and molecular mass of chitosan on the transfection efficiency. Biomaterials; 22:2075-2080.

(16) Mansouri S, Cuie Y, Winnik F, Shi Q, Lavigne P, Benderdour M et al. (2006). Characterization of folate-chitosan-DNA nanoparticles for gene therapy. Biomaterials; 27:2060-2065.

(17) Chellat F, Grandjean-Laquerriere A, Le N R, Fernandes J, Yahia L, Guenounou M et al. (2005). Metalloproteinase and cytokine production by THP-1 macrophages following exposure to chitosan-DNA nanoparticles. Biomaterials; 26:961-970.

(18) Corsi K, Chellat F, Yahia L, Fernandes J C (2003). Mesenchymal stem cells, MG63 and HEK293 transfection using chitosan-DNA nanoparticles. Biomaterials; 24:1255-1264.

(19) Liu F, Song Y, Liu D (1999). Hydrodynamics-based transfection in animals by systemic administration of plasmid DNA. Gene Ther, 6:1258-1266.

(20) Zhang G, Budker V, Wolff J A (1999). High levels of foreign gene expression in hepatocytes after tail vein injections of naked plasmid DNA. Hum Gene Ther; 10:1735-1737.

(21) Toumi H, Hegge J, Subbotin V, Noble M, Herweijer H, Best T M et al. (2006). Rapid intravascular injection into limb skeletal muscle: a damage assessment study. Mol Ther; 13:229-236.

(22) Mao H Q, Roy K, Troung-Le V L, Janes K A, Lin K Y, Wang Y et al. (2001). Chitosan-DNA nanoparticles as gene carriers: synthesis, characterization and transfection efficiency. J Control Release; 70:399-421.

(23) Xu X, Capito R M, Spector M (2008). Plasmid size influences chitosan nanoparticle mediated gene transfer to chondrocytes. J Biomed Mater Res A; 84:1038-1048.

(24) Lavertu M, Methot S, Tran-Khanh N, Buschmann M D (2006). High efficiency gene transfer using chitosan/DNA nanoparticles with specific combinations of molecular weight and degree of deacetylation. Biomaterials; 27:4815-4824.

(25) Danielsen S, Varum K M, Stokke B T (2004). Structural analysis of chitosan mediated DNA condensation by AFM: influence of chitosan molecular parameters. Biomacromolecules; 5:928-936.

(26) Mima S, Mararu M, Iwamoto R, Yoshikawa S (1983). Highly deacetylated chitosan and its properties. J Appl Polym Sci; 28:1909-1907.

(27) Chul C K, Hoon J J, Jung C H, Joe C O, Wan K S, Gwan P T (2005). Folate receptor-mediated intracellular delivery of recombinant caspase-3 for inducing apoptosis. J Control Release; 108:121-131.

(28) Kiang T, Wen J, Lim H W, Leong K W (2004). The effect of the degree of chitosan deacetylation on the efficiency of gene transfection. Biomaterials; 25:5293-5301.

(29) Hagstrom J E, Hegge J, Zhang G, Noble M, Budker V, Lewis D L et al. (2004). A facile nonviral method for delivering genes and siRNAs to skeletal muscle of mammalian limbs. Mol Ther; 10:386-398.

(30) Danialou G, Comtois A S, Matecki S, Nalbantoglu J, Karpati G, Gilbert R et al. (2005). Optimization of regional intraarterial naked DNA-mediated transgene delivery to skeletal muscles in a large animal model. Mol Ther, 11:257-266.

(31) Doh S G, Vahlsing H L, Hartikka J, Liang X, Manthorpe M (1997). Spatial-temporal patterns of gene expression in mouse skeletal muscle after injection of lacZ plasmid DNA. Gene Ther, 4:648-663.

### Other Embodiments

While specific embodiments have been described, those skilled in the art will recognize many alterations that could be made within the spirit of the invention, which is defined solely according to the following claims:

1. A polymer of Formula I: 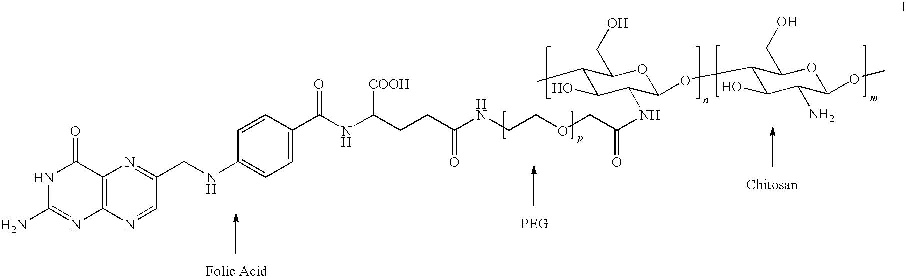
or a salt thereof,
wherein
n is an integer from 1 to 150, preferably 1 to 100, more preferably 1 to 50, more preferably 1 to 20, most preferably about 5 to 6,
m is an integer from 1 to 300, preferably 1 to 250, more preferably 1 to 200, most preferably about 167,
p is an integer from 1 to 200, preferably 1 to 150, more preferably 1 to 100, most preferably about 70; and
wherein the polymer is optionally labeled with a detectable label.

2. A drug delivery system, the system comprising a polymer according to Formula I: 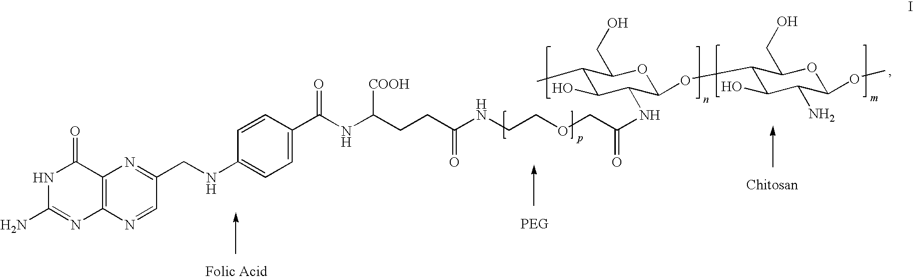
or a salt thereof
wherein
n is an integer from 1 to 150, preferably 1 to 100, more preferably 1 to 50, more preferably 1 to 20, most preferably about 5 to 6,
m is an integer from 1 to 300, preferably 1 to 250, more preferably 1 to 200, most preferably about 167,
p is an integer from 1 to 200, preferably 1 to 150, more preferably 1 to 100, most preferably about 70; and
wherein the polymer is optionally labeled with a detectable label.

3. A nanoparticle made of a polymer according to Formula I as defined in claim 1.

4. A drug delivery system for administration to a subject, the system comprising a nanoparticle made of a polymer according to Formula I as defined in claim 1, the nanoparticle comprising one or more therapeutic agents.

5. A method of treating a disease-state characterized by over-expression of folic acid receptors on a cell surface, the method comprising: administering to a subject in need thereof, a therapeutically effective amount of a polymer according to Formula I as defined in claim 1, so as to treat the disease.

6. A method of treating a disease-state characterized by over-expression of folic acid receptors on a cell surface, the method comprising: administering to a subject in need thereof, a drug delivery system containing a therapeutically effective amount of a polymer according to Formula I as defined in claim 1 so as to treat the disease.

7. The method of claim 5 wherein the disease is an autoimmune disease.

8. The method of claim 7 wherein the autoimmune disease is rheumatoid arthritis.

9. The method of claim 6 wherein the disease is an autoimmune disease.

10. The method of claim 9 wherein the autoimmune disease is rheumatoid arthritis.


SEQUENCE LISTING (2 sequences)

SEQ ID NO: 1 - DNA, 29 bp, artificial sequence
  oligonucleotide
ccaagcttgaatggaaatctgcagaggcc

SEQ ID NO: 2 - DNA, 31 bp, artificial sequence
  oligonucleotide
gctctagactgggcagtactactcgtcctcc